(19) United States
(12) Patent Application Publication
(10) Pub. No.: US 2022/0362701 A1
(43) Pub. Date: Nov. 17, 2022

(54) Air Purification and Decontamination Devices, Systems, and Methods

(71) Applicant: Paul Chirayath, Lockport, NY (US)

(72) Inventor: Paul Chirayath, Lockport, NY (US)

(21) Appl. No.: 17/663,251

(22) Filed: May 13, 2022

Related U.S. Application Data
(60) Provisional application No. 63/187,978, filed on May 13, 2021.

(51) Int. Cl. B01D 46/00; B01D 39/16; B01D 46/24; A61L 9/20; A61L 9/015

(52) U.S. Cl. CPC B01D 46/0028; B01D 39/1623; B01D 46/0049; B01D 46/2411; A61L 9/20; A61L 9/015; B01D 2279/65; B01D 2273/30; B01D 2279/50; A61L 2209/14; A61L 2209/212

(57) ABSTRACT

Air purification devices include an air inlet and outlet, upstream and downstream sections in air flow communication with the air inlet and outlet and a filter removably positioned between the upstream and downstream sections to filter at least pathogens from air being passed through the filter. One or more ultraviolet (UV) illuminating sources may be positioned in at least the upstream section proximate the filter to illuminate at least an upstream surface of the filter with sufficient UV radiation to kill viable pathogens that accumulate on the filter. One or more ozone generating sources may be positioned to generate ozone in sufficient concentrations to kill viable pathogens proximate the device. The UV illuminating sources and ozone generating sources may be positioned to provide a predetermined pressure drop across the sources and air flow pattern proximate the filter, and enable the filter to be removed without handling the sources.

### CROSS-REFERENCE TO RELATED APPLICATIONS

This application claims priority to and the benefit of U.S. Provisional Patent Application No. 63/187,978 filed on May 13, 2021, which is incorporated by reference in its entireties.

### STATEMENT REGARDING FEDERALLY SPONSORED RESEARCH OR DEVELOPMENT

(Not Applicable)

### BACKGROUND OF THE INVENTION

### Field of the Invention

The present invention generally relates to air filtration. More specifically, the invention relates to air purification devices, systems, and methods and decontamination thereof.

### Background Art

The continuing development of high efficiency particulate air (HEPA) filter technologies continues to expand the applications and availability of air purification systems. The development of mobile HEPA filtration systems has further expanded the availability of this technology.

One unfortunate consequence of being able to filter particulates down to the size of viruses, bacteria, and other pathogens (collectively, “pathogens”) is that the HEPA filter themselves may become contaminated with the pathogens removed from the air. As such, care must be taken with these filters to ensure that particulates trapped by the filter are not released to the air during the operation and maintenance of the HEPA filter system.

Various containment and decontamination technologies have been developed for disposing of and/or decontaminating the HEPA filtration devices and systems. For example, various filter media have been developed specifically for retaining pathogens. The filter material may also be coated and/or impregnated with materials to improve the retention of and/or kill pathogens in contact with the material.

Various devices and systems have also implemented ultraviolet (UV) illumination and ozone technologies to decontaminate the device or room as both technologies are known to kill pathogens at sufficient intensity and concentrations and exposure times. See, for example, U.S. Pat. No. 7,326,387, on which the present inventor is listed as a co-inventor.

The global pandemic resulting from COVID-19 has dramatically increased the demand and performance levels of these devices. As such, there is a continuing need for air purification devices, systems, and methods and decontamination techniques with higher performance to support the expanding global need for these devices, systems, and methods.

### BRIEF SUMMARY OF THE INVENTION

The present invention addresses the above noted needs by providing improved air purification and decontamination devices, systems, and methods that enable more effective ultraviolet illumination, ozone generation, and operation and maintenance of the devices and systems.

Devices and systems provide for continuous or intermittent ultraviolet (UV) illumination of an upstream and/or downstream facing surface of a HEPA filter during operation and/or shutdown to reduce the concentration of viable pathogens that accumulate on, in or proximate the filter, the device, and system to non-hazardous concentrations, which is preferably zero or very close to it. Furthermore, the UV light may be emitted by one or more illuminating sources positioned at a distance away from the filter surface to enable 1) the UV light to illuminate all or a desired portion of the filter surface to substantially reduce the concentration of, or preferably eliminate, viable pathogens, i.e., kill pathogens, that accumulate on, in or proximate the filter, and 2) the removal of the filter from the device or system without having to remove or otherwise handle the illuminating UV sources. In various embodiments, at least some of the UV sources emit UV light in the 254 nm range, which is shown to be germicidal to pathogens.

In various embodiments, some or all of the ductwork proximate to the UV illuminating sources on the inlet/upstream section and/or outlet/downstream section of the device is composed of, or coated with, a material that is reflective of UV radiation. In addition, the ductwork may be shaped such that UV light reflected from the ductwork is directed toward the filter surface.

One or more ozone generating sources may be employed in the upstream and/or downstream sections of the device and positioned to generate ozone in sufficient concentrations within the device and/or room for sufficient lengths of time to kill pathogens, or substantially reduce the concentration of viable pathogens, in or on the device and room/area to non-hazardous concentrations, which is preferably zero or very close to it. For example, one or more UV sources emitting UV light in the 182 nm range may be employed to generate ozone in sufficient concentrations to be germicidal. As with the illuminating UV source, the ozone generators may be positioned at a distance away from the filter surface to enable 1) the generation of ozone in sufficient concentration at the filter, and 2) the removal of the filter from the device or system without having to remove or otherwise handle the ozone generating sources.

In various embodiments, the illuminating and ozone generating sources are positioned and/or shaped to 1) reduce the pressure drop introduced into the air flow path by the source, and 2) support air flow through all or desired portions of the filter. For example, upstream of the filter, it may be desirable to position the sources a sufficient distance from the filter to allow relatively uniform air flow distribution across and through the entire filter surface, while being sufficiently close to the filter to substantially reduce viable pathogens, i.e., kill pathogens, that accumulate on, in, or proximate the filter, as well as the proximate the device 10. Downstream of the filter, the sources may be positioned to provide a pressure drop and flow pattern conducive to achieving desired flow pattens upstream of the filter.

In various embodiments, the device and system may include an upstream and/or downstream air flow duct and the filter may be sized from a cross-sectional area perspective to provide a desired flow rate and pressure drop through the filter. For example, when devices of the present invention are implemented as part of a building air handling system, e.g. heating, ventilation/circulation, air conditioning, etc., it may be desirable to increase the cross-sectional area of the upstream and/or downstream duct and the filter to support air flow rates through the building system. Alternatively, the devices may be employed in parallel to enable maintenance of one device while other devices are operational, as well as in stages as may be desired. When the devices are operated as a stand-alone unit, either stationary or mobile, the cross-sectional areas and fan design may be adjusted to achieve desired design parameters, such as air flow, noise level, etc.

Accordingly, the present disclosure addresses the continuing need for air purification devices, systems, and methods with improved cost and performance.

### BRIEF DESCRIPTION OF THE DRAWINGS

The accompanying drawings are included for the purpose of exemplary illustration of various aspects of the present invention, and not for purposes of limiting the invention, wherein:

FIG. 1 depicts exemplary exterior views of stand-alone air purification and decontamination device and system embodiments.

FIG. 2 depicts exemplary exterior views of air purification and decontamination devices integrated with building air circulation system embodiments.

FIG. 3 depicts exemplary cross-sectional views of air purification and decontamination device and system embodiments.

FIG. 4A depicts a perspective view of various embodiments of the device.

FIG. 4B depicts various embodiments such as those shown in FIG. 4A with an air inlet cover removed.

FIG. 4C depicts exemplary embodiments of an inlet/upstream section of the device.

FIG. 4D depicts exemplary embodiments of an outlet/downstream section of the device.

FIG. 5 depict a perspective view of various embodiments of devices and systems.

FIG. 6 illustrates exemplary components of computing and communication resources.

FIG. 7 shows a picture of the experimental device along with the input conditions and output results from the testing.

FIG. 8 shows air sampling results detecting pathogens from before, during, and after operating the experimental device.

FIGS. 9-10 depict exploded views of various embodiments of the device.

FIGS. 11A & 11B depict assembled and exploded views of various embodiments of the device.

FIGS. 12A & 12B depict assembled and exploded views of various embodiments of the device.

In the drawings and detailed description, the same or similar reference numbers may identify the same or similar elements. It will be appreciated that the implementations, features, etc. described with respect to embodiments in specific figures may be implemented with respect to other embodiments in other figures, unless expressly stated, or otherwise not possible.

### DETAILED DESCRIPTION OF THE INVENTION

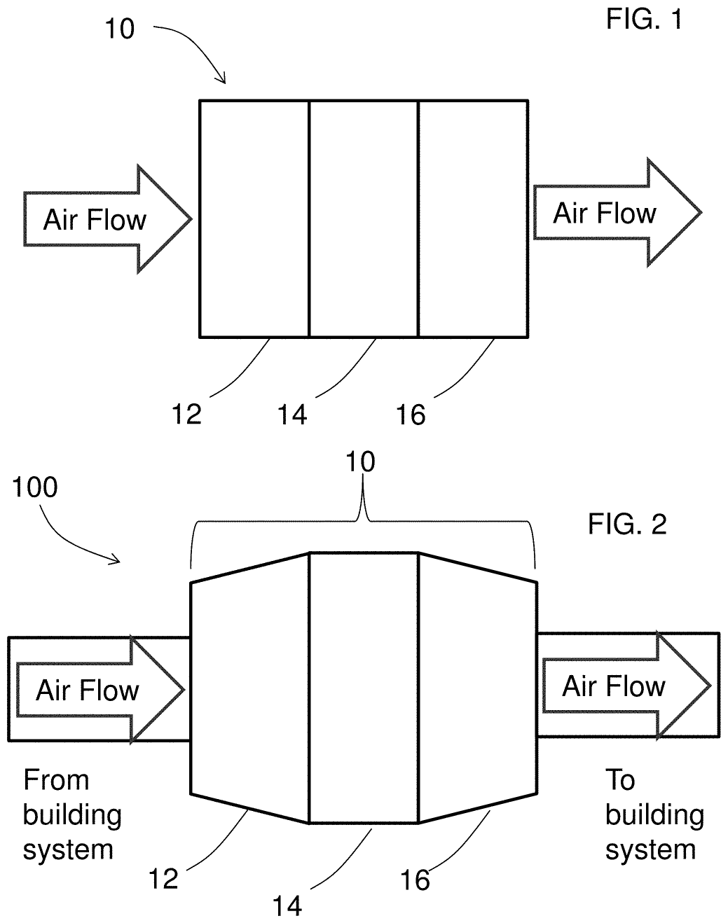

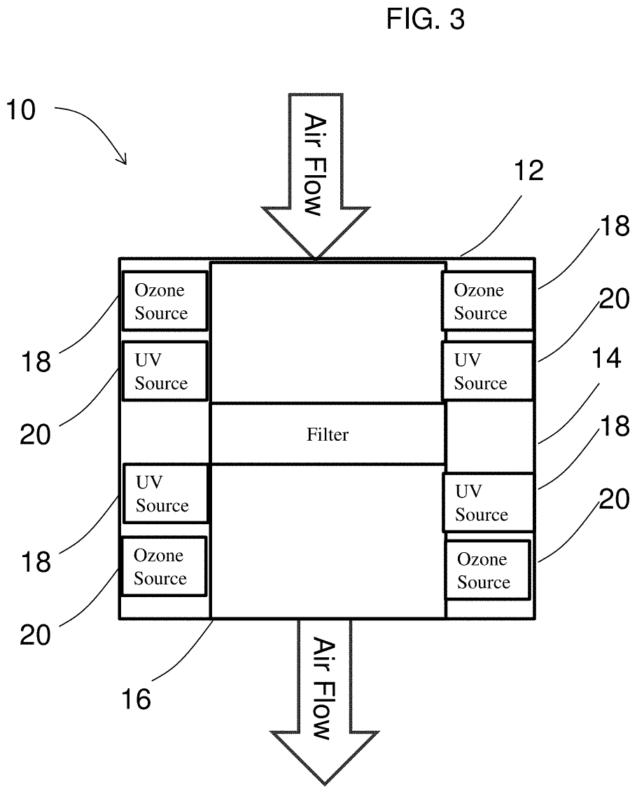

Devices 10 of the present invention may be employed as a stand-alone system or as part of a building air circulation system 100 as shown in FIGS. 1 and 2, respectively. The devices 10 generally include an air inlet/upstream duct section 12 upstream of a filter 14 and an air outlet/downstream duct section 16 downstream of the filter 14. It will be appreciated that the terms inlet, outlet, upstream, and downstream are relative to the direction of air/gas flow through the device 10. Also, while the description is generally set forth referencing air, the system 10 may be used with other gases. The system 100 may be a building air circulation system associated with heating, ventilation, air conditioning, etc. The device 10 will generally include various fans, controls, power sources, sensors, etc. as may be needed to operate the device 10 in a desired configuration.

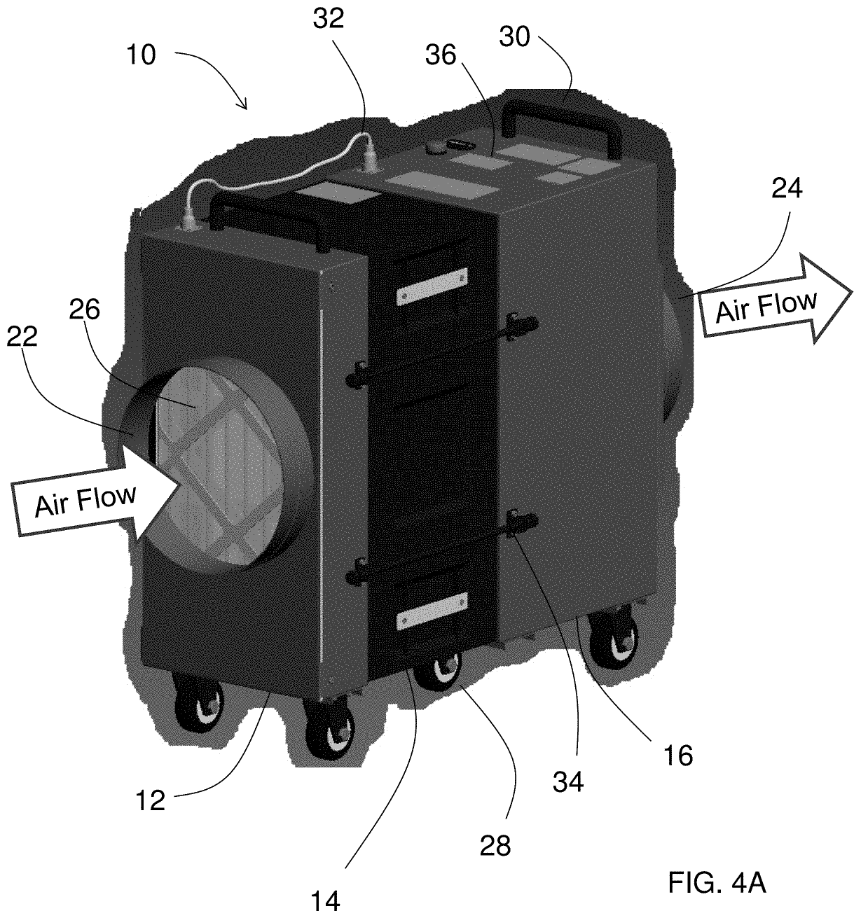

FIG. 3 depicts cross-sectional views of air purification and decontamination device and system embodiments 10. The device 10 may include one or more the UV illuminating sources 18 and/or ozone generating sources 20 and associated hardware that have been substantially removed from the air flow path and positioned to enable the filter 14 to be accessed and replaced without handling the sources 18 or 20 and providing for a substantially uniform air flow pattern across the filter 14, while effectively reducing viable pathogen, i.e, killing pathogens, concentrations on, in, or proximate the filter 14 to concentrations that are not considered hazardous.

Furthermore, the separation of the sources 18 and 20 from the proximity of the filter 14 enables standard form factor filters to be used and enables more straightforward procedures to be employed in the maintenance and replacement of the filter 14. The filter 14 may include an upstream surface facing the upstream section 12, a downstream surface 16 facing the downstream section 16 and an interior between the upstream and downstream surfaces that may be exposed to UV light and ozone used to reduce viable pathogens, i.e., kill pathogens, in, on, or proximate the filter 14 to non-hazardous concentrations, which is preferably zero or very close to it.

It will be appreciated that FIG. 3 depicts exemplary embodiments regarding the positioning of the filter 14 and the sources 18 and 20. The view may be a vertical or horizontal cross-section depending upon the embodiment. The UV illuminating sources 18 and ozone generating sources 20 are depicted on both side of the cross-section and both on the upstream side 12 and downstream side 16 of the filter 14, but may be on 1-4 sides with some sources 18 and 20 being on the same or different sides.

It will be appreciated that the sources 18 and 20 may be positioned in the air flow path and/or around the periphery of the upstream and downstream air ducts, 12 and 16, as desired to achieve a desired irradiation pattern of the filter 14 and air flow pattern through the filter 14. As further discussed herein, the number and location of the sources 18 and 20 may be decided by the skilled practitioner to suit a particular implementation; however, it is generally preferable to position the sources 18 and 20 so as to a) not create dead or low exposure zones in terms of 1) UV illumination of the filter 14, and 2) air flow rate, and b) enable the filter 14 to be removed from the device 10 without handling the sources 18 and 20.

Since the majority of the contaminants removed by the filter 14 may be present on the upstream side 12 of the filter 14 in many embodiments, it may be desirable to deploy at least some of the UV illuminating sources 18 in the upstream side 12 of the filter 14 in the device 10. Conversely, since ozone can pass through the filter 14, it may be desirable to position the ozone generating sources 20 in the downstream side 16 of the device 10, so as to not increase the pressure drop or flow pattern disturbance on the upstream side 12 of the device 10. In certain environments such highly toxic or lethal contaminants, it may be preferable to deploy ozone generating sources 20 in both the upstream 12 and downstream 16 sections of the device 10.

In various embodiments, the UV illuminating source 18 may be a commercial off the shelf or custom UV emitter that emits UV light in the 254 nm wavelength range, which is known to have a germicidal effect on pathogens. The number of UV illuminating sources 18 employed in the device 10 may be decided by one of ordinary skill to achieve a desired objective. Studies have found that UV light at 254 nm emitted by a 30 mW source is effective at killing pathogens from 1 meter (see, Kowalski, W. J., Bahnfleth, W. P.; “UVGI Design Basics for Air and Surface Disinfection”; HPAC Engineering, January 2000, v72n1, :100-110). However, since an objective is to kill as much of the pathogen as possible as quickly and efficiently as possible without impacting the operation of the device 10, then it may be desirable to 1) position the UV illuminating source(s) 18 proximate the upstream side surface of the filter 14 (e.g., <1 inch), but not sufficiently close to create illumination and/or air flow shadows or dead zones, and 2) employ high power one or more UV sources to enable as much as 10,000 mW intensity to increase the likelihood of disinfection. Other UV sources, such as LED sources, that emit wavelengths that are germicidal to pathogens may be employed as well.

The ozone generator source 20 may be embodied in various forms suitable for the generation of ozone in sufficient quantities to kill pathogens. Studies have shown that ozone concentration levels in the 3-4 ppm range are effective to kill pathogens (see Kowalski, W. J., Bahnfleth, W. P., and Whittam, T. S.; “Bactericidal Effects of High Airborne Ozone Concentrations on Escherichia coli and Straphylococcusaureus”, Ozone Science & Engineering, 1998, v20, :205-221.)

Ozone may be generated using standard methods, such as via electrical current or UV light. For example, a UV light source emitting UV radiation in the 182 nm range may be used to generate ozone in sufficient quantities to reach effective germicidal concentrations within the device 10 or room. Other sources, such as LED sources, that emit wavelengths that generate ozone may also be employed. The number and power of the ozone generating sources 20 may be determined by the skilled artisan depending upon the size of the device 10 or room being implemented and the specific application.

While the sources 18 and 20 are depicted in FIG. 3 on the periphery of the air ducts 12 and 16, the sources 18 and 20 may be positioned partially or fully within the air ducts 12 or 16 to achieve a specific irradiation pattern. However, the positioning of the sources 18 and 20 in the air flow path will introduce additional pressure drop into the device 10, which may in turn increase motor size requirements and the level of operational noise from the device 10. In addition, as previously noted, the introduction of the sources 18 and 20 into the air flow path will impact the air flow pattern at the filter 14. As such, it may be desirable depending upon the flow rate and design of the remainder of the upstream air flow to position sources 18 or 20 that are placed in the air flow stream to be sufficient distance from the filter 14 to allow the air flow pattern to recover from the obstruction presented by the sources 18 or 20 prior to encountering the upstream side of the filter 20. In other implementations, the system requirements for the device 10 may established to take into effect high and low air (flow) zones resulting from the presence of the sources 18 and 20 in the air flow path.

It will be appreciated that the pressure drop and flow pattern introduced by the positioning of the sources 18 and 20 and the illumination of the filter by UV radiation may be balanced to achieve design objectives. For example, minimizing the pressure drop and flow pattern disturbance of the sources 18 and 20 in the air flow stream may not result in an optimal UV illumination of the filter 14. Conversely, maximizing the illumination of the filter 14 may not result in optimal pressure drop and flow patterns through the filter 14.

FIG. 3 further depicts the sources 18 and 20 as separate sources. It will be appreciated that the sources 18 and 20 may be housed together and it may be possible to have a UV emitter that serves as an illuminating source 18 and an ozone generator source 20.

In various embodiments, the upstream and/or downstream air duct sections, 12 and 16, may be constructed and/or coated, in whole or part, with materials that reflect the UV light emitted by the UV illuminating and/or ozone generating sources 18 or 20 to increase the amount of UV radiation that contacts the filter 14. See, for example, aluminum and e-PTFE are reflective of UV light in the 250-280 nm range, which include UV wavelengths typically suitable for pathogen disinfection. It will be further appreciated that it may be desirable to employ materials or coat some or all surfaces near the inlet and/or exit of the device 10 that absorb UV light, as well as breakdown ozone, particularly for devices 10 that are not employed in a closed air circulation system.

In various embodiments, the filter 14 is removable from the device 10 without removing or otherwise handling the sources 18 and 20. The device 10 may be designed to enable commercial off the shelf filters to be used. The internal structure of the filter and/or the number of filter stages may be varied by the skilled artisan to achieve various pressure drops and particle size filtration characteristics. For example, it may be desirable to use a filter 14 made of fiberglass. In various embodiments or applications, it may be desirable to impregnate the filter 14 with a germicidal material.

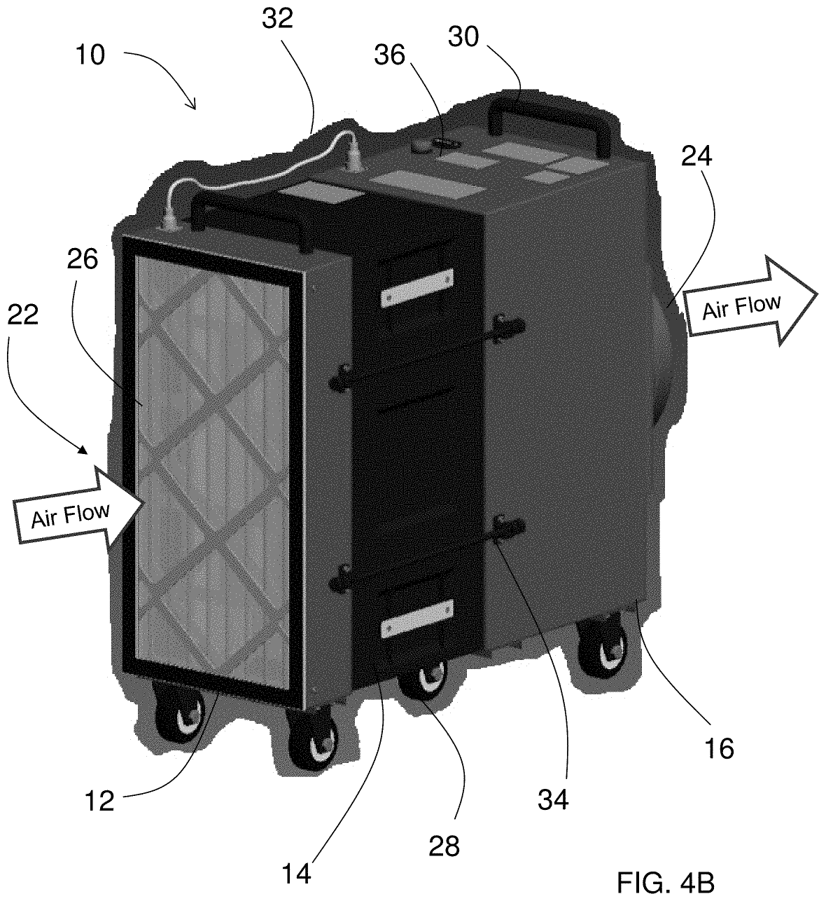

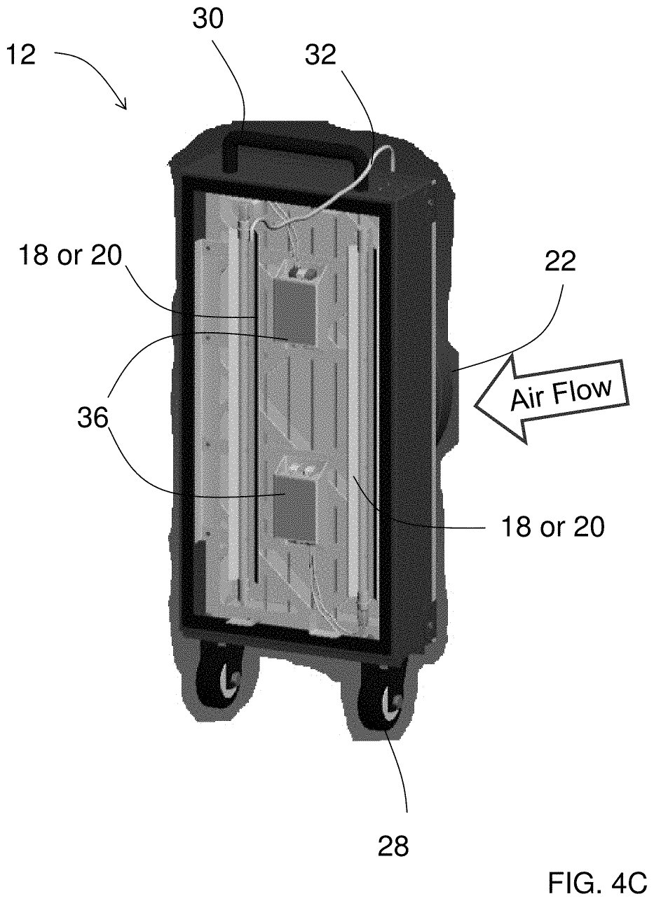

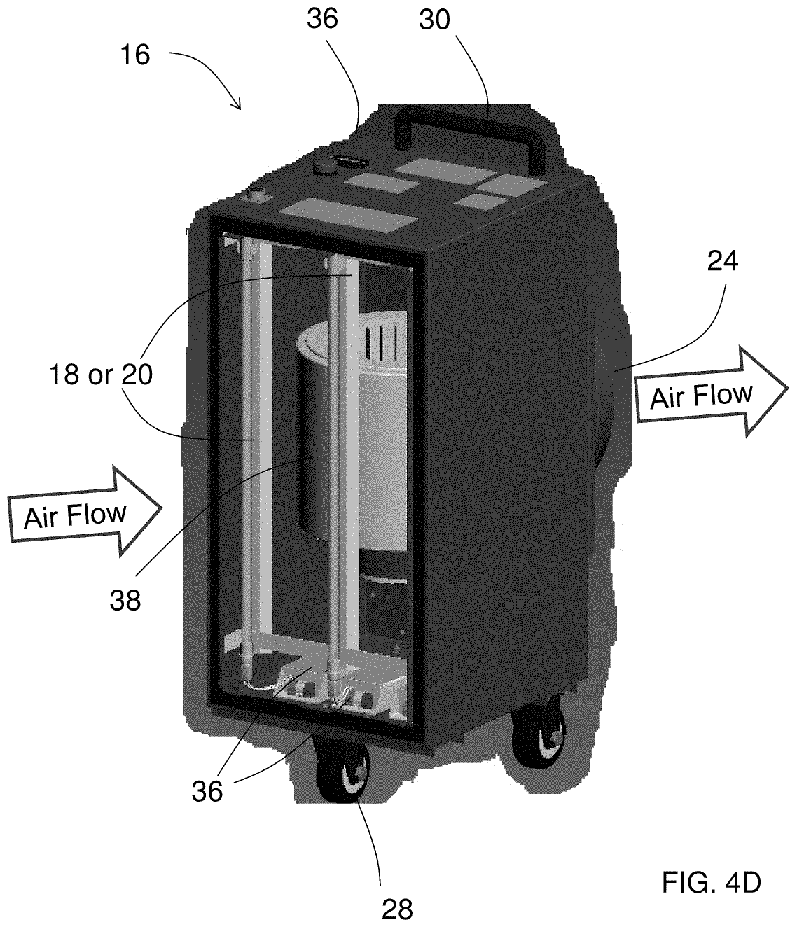

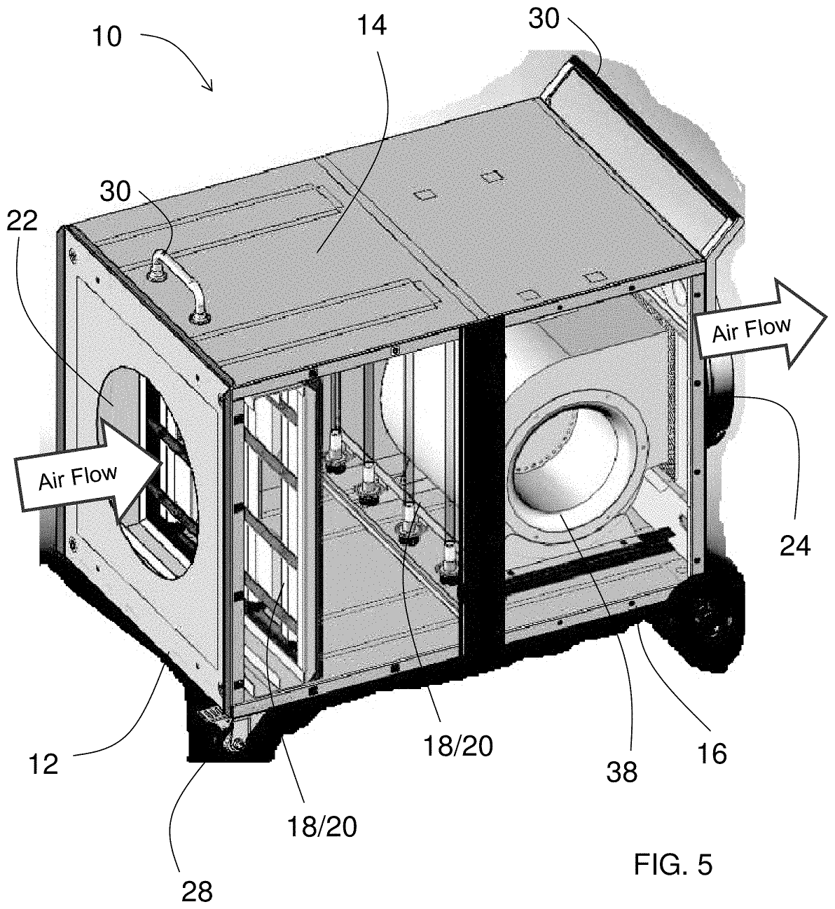

FIG. 4A depicts a perspective view of various embodiments of the device 10 as a stand-alone system. As depicted, air is drawn into the device 10 via one or more fans (see FIG. 4C) via an air inlet 22 and exhausted via an outlet 24 (FIG. 4D). The device 10 may include a course inlet filter 26 to filter large particulate matter, such as lint and dust, before it reaches the HEPA filter 14. While the course inlet filter is not necessary to the invention, it will increase the useful life of the HEPA filter 14 by removing large particulate matter, which, due to the coarseness, typically will not concentrate pathogens on the filter 26. That be said, the present invention may be implemented such that ozone generated by the device 10 may be of sufficient concentration to kill pathogens captured by the course inlet filter 26 or in the surrounding environment or room. The device 10 may be configured to allow the course inlet filter 26 to be easily replaced and/or removed and cleaned.

Mobile devices 10 may include wheels 28, handles 30, one or more power supplies, which may include a wall plug (not shown), and other functional elements required to assemble and operate the device 10. Power may be supplied to the upstream section 12 and/or the downstream side 16 of the device 10. In various embodiments, power is supplied from the downstream 16 to the upstream side 12 via a power cable 32, which may be external, as shown in FIG. 4A, or internal to the device 10. The power cable 32 may also include control wiring to allow signaling between controllers and other components in the upstream and downstream sections of the device 10. An external power cable 32 may be useful if the device 10 is separable into upstream 12, downstream 16, and filter 14 sections.

In various embodiments, the filter section 14 may be completely separable from the upstream 12 and downstream 16 sections of the device 10. In this manner the filter section 14 may be replaced and/or removed and cleaned in its entirety. For example, in FIG. 4A, the filter section 14 may be sandwiched between the upstream section 12 and the downstream section 16 and secured with one or more fasteners 34.

The device 10 may include one or more controllers 36, which may include one or more control panels and displays to enable user monitoring and control at the device 10. Wired or wireless control and user interfaces may be provided as well by the skilled artisan to enable various types of remote and/or proximate control and monitoring. The controllers 36 may be configured to control the operation of the sources 18 and 20, fans 38 (see FIGS. 4D & 5), etc. and receive input from various sensors 37 detecting various parameters, such as air flow rate, ozone concentration, UV light intensity, temperature, pressure drop, noise levels, etc.

FIG. 4B depict various embodiments such as those shown in FIG. 4A with an air inlet 22 cover removed to make the course inlet filter 26 more visible in the drawing.

FIG. 4C depicts the inlet/upstream section 12 of the device 10. The upstream section 12 may include UV illuminating sources 18, as well as ozone generating sources 20, as well as controllers 36 for the sources and in communication with the control panel.

FIG. 4D depicts the outlet/downstream section 16 of the device 10. The downstream section 16 may include UV illuminating sources 18, as well as ozone generating sources 20, as well as controllers 36 for the sources and in communication with the control panel.

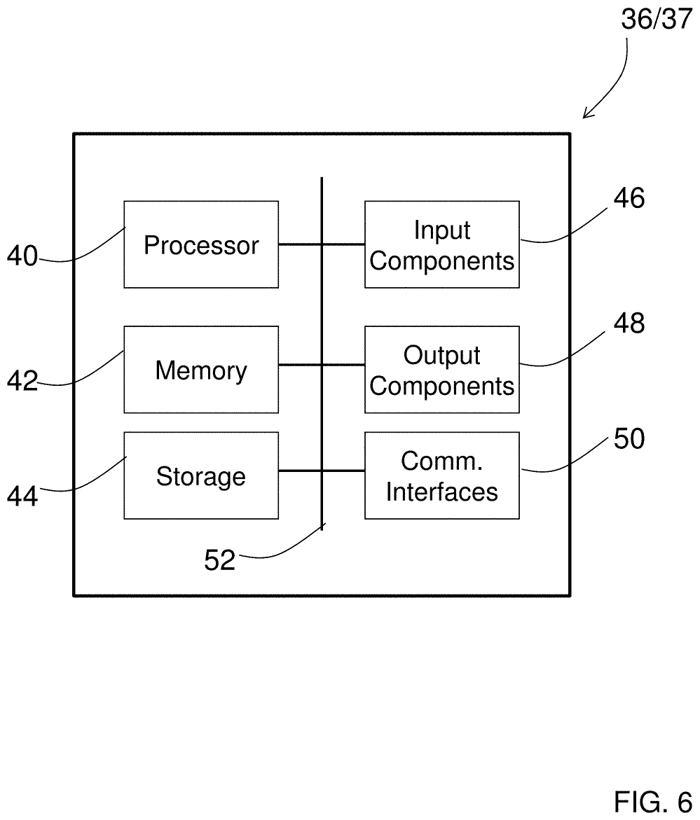

FIG. 5 depict a perspective view of various embodiments with the outer shell/enclosure removed to show various features. FIG. 5 embodiments may include an integrated chassis that may be used with a cartridge filter 14 supported by the chassis.

In various embodiments, such as those depicted in FIGS. 4A-5, it may be desirable to provide UV illuminating sources 18 in the upstream section 12 and ozone generating UV sources 20 in the downstream section 16.

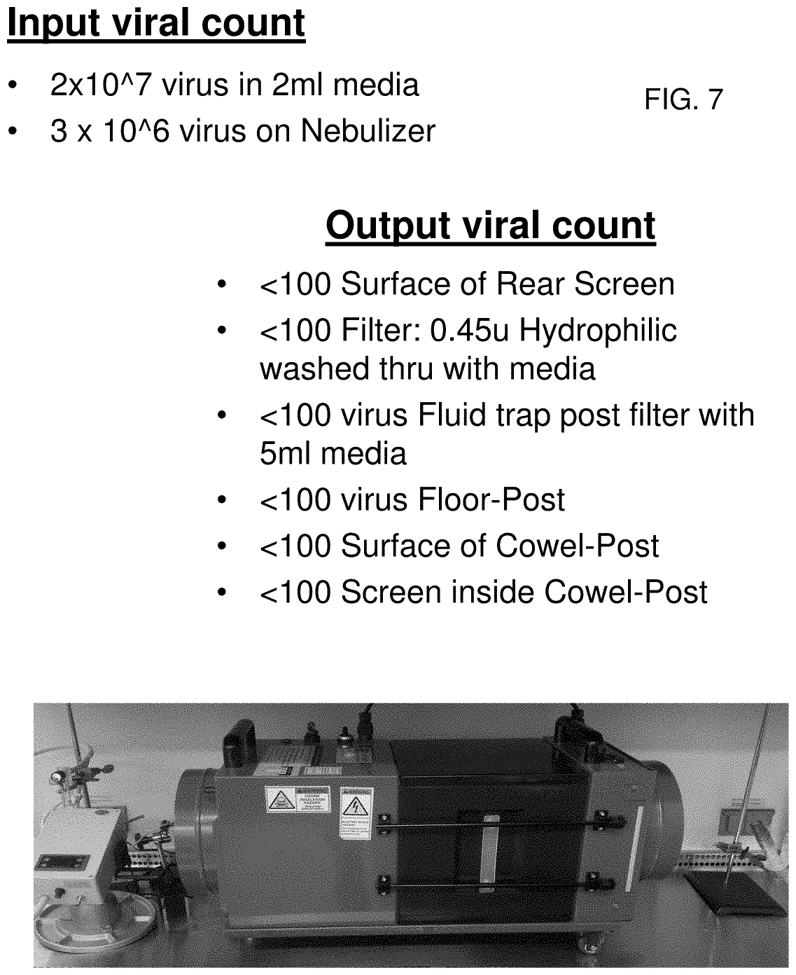

FIG. 6 illustrates exemplary component embodiments of various computing resources that may be employed in the controllers 36, sensors 37, and elsewhere in the device 10, to perform various functions, gather and communicate data, and run various applications. The computing resources may each include one or more processors 40, memory 42, storage 44, input components 46, output components 48, communication interfaces 50, as well as other components that may be interconnected as desired by the skilled artisan via one or more buses 52. As previously described, the components of the various computing resources may often be configured as a single device or multiple interdependent or stand-alone devices in close proximity and/or distributed over geographically remote areas.

Processor(s) 40 may include one or more general or Central Processing Units (“CPU”), Graphics Processing Units (“GPU”), Accelerated Processing Units (“APU”), microprocessors, and/or any processing components, such as a Field-Programmable Gate Arrays (“FPGA”), Application-Specific Integrated Circuits (“ASIC”), etc. that interpret and/or execute logical functions. The processors 40 may contain cache memory units for temporary local storage of instructions, data, or computer addresses and may be implemented as a single-chip, multiple chips and/or other electrical components including one or more integrated circuits and printed circuit boards that implements and executes logic in hardware, in addition to executing software.

Processor(s) 40 may connect to other computer systems and/or to telecommunications networks as part of performing one or more steps of one or more processes described or illustrated herein, according to particular needs. This can be accomplished through APIs or other methods, using FHIR format or other health-specific format. Moreover, one or more steps of one or more processes described or illustrated herein may execute solely at the processor 40. In addition, or as an alternative, one or more steps of one or more processes described or illustrated herein for execution in one processor may be executed at multiple CPUs that are local or remote from each other across one or more networks.

The computing resources of the system 10 may implement processes employing hardware and/or software to provide functionality via hardwired logic or otherwise embodied in circuits, such as integrated circuits, which may operate in place of or together with software to execute one or more processes or one or more steps of one or more processes described or illustrated herein. Software implementing particular embodiments may be written in any suitable programming language (e.g., procedural, object oriented, etc.) or combination of programming languages, where appropriate.

Memory 42 may include Random Access Memory (“RAM”), Read Only Memory (“ROM”), and/or another type of dynamic or static storage device, such as flash, magnetic, and optical memory, etc. that stores information and/or instructions for use by processor 40. The Memory 42 may include one or more memory cards that may be loaded on a temporary or permanent basis. Memory 42 and Storage 44 may include a Subscriber Identification Module (“SIM”) card and reader.

Storage components 44 may store information, instructions, and/or software related to the operation of the system 10 and computing resources. Storage 44 may be used to store operating system, executables, data, applications, and the like, and may include fast access primary storage, as well as slower access secondary storage, which may be virtual or fixed.

Storage component(s) 44 may include one or more transitory and/or non-transitory computer-readable media that store or otherwise embody software implementing particular embodiments. The computer-readable medium may be any tangible medium capable of carrying, communicating, containing, holding, maintaining, propagating, retaining, storing, transmitting, transporting, or otherwise embodying software, where appropriate, including nano-scale medium. The computer-readable medium may be a biological, chemical, electronic, electromagnetic, infrared, magnetic, optical, quantum, or other suitable medium or a combination of two or more such media, where appropriate. Example computer-readable media include, but are not limited to fixed and removable drives, ASIC, Compact Disks (“CDs”), Digital Video Disks (“DVDs”, FPGAs, floppy disks, optical and magneto-optic disks, hard disks, holographic storage devices, magnetic tape, caches, Programmable Logic Devices (“PLDs”), RAM devices, ROM devices, semiconductor memory devices, solid state drives, cartridges, and other suitable computer-readable media.

Input components 46 and output components 48 may include various types of Input/Output (“I/O”) devices. The I/O devices often may include a Graphical User Interface (“GUI”) that provides an easy to use visual interface between the user and system 10 and access to the operating system or application(s) running on the devices.

Input components 46 receive any type of input in various forms from users or other machines, such as touch screen and video displays, keyboards, keypads, mice, buttons, track balls, switches, joy sticks, directional pads, microphones, cameras, transducers, card readers, voice and handwriting inputs, and sensors for sensing information such as biometrics, temperature & other environmental conditions, such as air quality, etc., location via Global Positioning System (“GPS”) or otherwise, accelerometer, gyroscope, compass, actuator data, which may be input via a component in the user device 10 and/or received via one or more communication interfaces 50.

Output components 48 may include displays, speakers, lights, sensor information, mechanical, or other electromagnetic output. Similar to the input, the output may be provided via one or more ports and/or one or more communication interfaces 50.

Communication interfaces 50 may include one or more transceivers, receivers, transmitters, modulators, demodulators that enable communication with other devices, via wired and/or wireless connections. Communication interface 30 may include Ethernet, optical, coaxial, Universal Serial Bus (“USB”), Infrared (“IR”), Radio Frequency (“RF”) including the various Wi-Fi, WiMax, cellular, and Bluetooth protocols, such as Bluetooth, Bluetooth Low Energy (BLE), Wi-Fi (IEEE 802.11), Wi-Fi Direct, SuperWiFi, 802.15.4, WiMax, LTE systems, LTE Direct, past, current, and future cellular standard protocols, e.g., 4-5G, or other wireless signal protocols or technologies as described herein and known in the art.

Bus(es) 52 may connect a wide variety of other subsystems, in addition to those depicted, and may include various other components that permit communication among the components in the computing resources. The Bus(es) 52 may encompass one or more digital signal lines serving a common function, where appropriate, and various structures including memory, peripheral, or local buses using a variety of bus architectures. As an example and not by way of limitation, such architectures include an Industry Standard Architecture (“ISA”) bus, an Enhanced ISA (“EISA”) bus, a Micro Channel Architecture (“MCA”) bus, a Video Electronics Standards Association Local Bus (“VLB”), a Peripheral Component Interconnect (“PCI”) bus, a PCI-eXtended (“PCI-X”) bus, a Peripheral Component Interconnect Express (PCIe) bus, a Controller Area Network (“CAN”) bus, and an Accelerated Graphics Port (“AGP”) bus.

The computing resources of the system 10 may provide functionality as a result of the processors 40 executing software embodied in one or more computer-readable storage media residing in the Memory 42 and/or Storage 44 and logic implemented and executed in hardware. The results of executing the software and logic may be stored in the Memory 42 and/or Storage 44, provided to output components 48, and transmitted to other devices via communication interfaces 50, which includes cloud storage and cloud computing. In execution, the processor 40 may use various inputs received from the input components 46 and/or the communications interfaces 50. The input may be provided directly to the processor 40 via the Bus 52 and/or stored before being provided to the processor 40. Executing software may involve carrying out processes or steps may include defining data structures stored in Memory 42 and modifying the data structures as directed by the software.

An experimental device 10 of the present invention was constructed for testing to determine the air purification characteristics. The experimental devices 10 are exemplary of the embodiments shown in FIGS. 4A-5, in which two UV illuminating sources 18 were deployed in both the upstream section 12 and downstream section 16 of the device 10 and three UV ozone generating sources 20 were deployed in the downstream section 16 of the device 10. The filter had a pore size ranging from 0.1 to 0.45 micron, and fan operated at 2.0 L/min.

Since SARS-CoV2 requires BSL-3 facility, testing was performed using OC43, a human beta-coronavirus, in a BSL-2 enclosure using a relevant pulmonary epithelial cell A-549 to count infectious virus. RNA only was detected using a reverse transcription polymerase chain reaction (RT-PCR) technique.

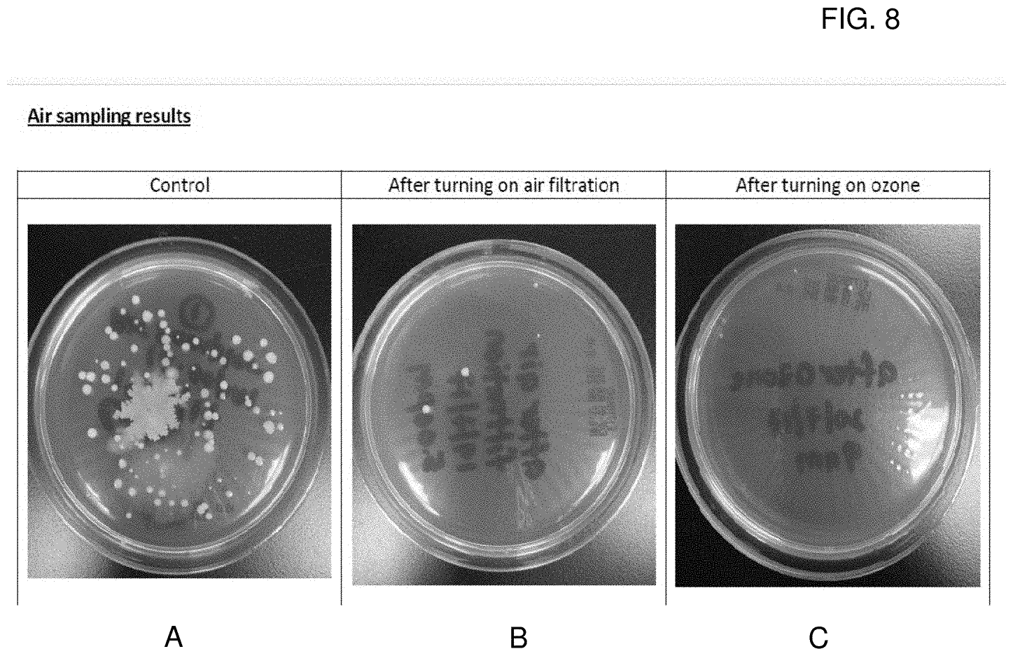

FIG. 7 shows a picture of the experimental device 10 along with the input conditions and output results from the testing. The results show a dramatic reduction (from 2×10⁷ to less than 10²) in the virus count between the inlet and outlet of the device 10 during operation.

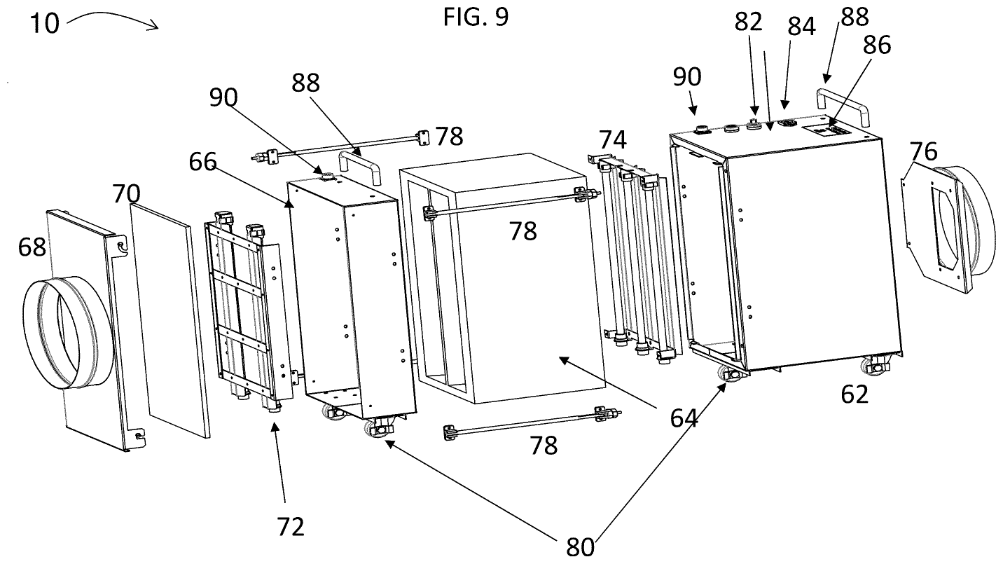

FIG. 8 shows air sampling results detecting pathogens from before, during, and after operating the experimental device 10, such as shown in FIG. 7. Picture A is air sampling prior to operation of the device 10. Picture B is air sampling during operation of the device 10. Picture C is air sampling after air filtration has been performed and ozone was generated by the device 10. As can be seen by the sample, operating the device 10 significantly reduced the pathogens detected in the air.

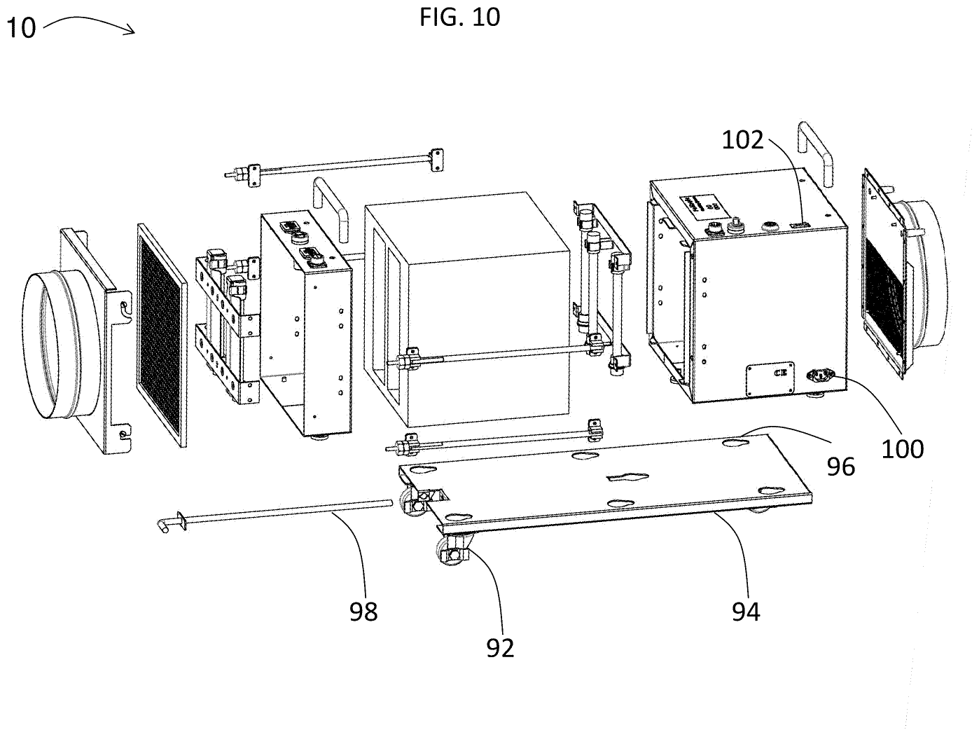

FIG. 9 depicts an exploded view of APDD 10 embodiments having 3 (three) main structures: rear air processing plenum 62, HEPA filter 64, front UVC air entry plenum 66. Air may enter via an adapter 68, which may be removable duct, pass through one or more prefilters 70, which may employ various materials, such as stainless steel, paper fiber, etc. (not shown) over a UVC lamp reflector assembly 72. The air may be directed through the HEPA filter 64, which may be one or more V-banks, and passed through a rear UVC ozone lamp cage 74 and the rear air processing plenum 62 before being exhausted, such as via an air duct adapter 76, which may be removable.

Other components may include: tensions rods 78 (4) to clamp the HEPA filter 64 between the front 66 and back 62 air plenums, wheels 80, motor speed control 82, non-resettable and resettable hour meters 84 (2), LED machine operating monitor 86, handles 88, power transfer sockets 90.

FIG. 10 depicts an exploded view of other embodiments of APDD, similar to those shown in FIG. 9. The operation and method of construction may be the same or similar the embodiments of FIG. 9, but a smaller size and air flow capability. For smaller sizes, wheels 92 may be provided that attach to a removable base 94 to facilitate lag mounting in a room or vehicle such as an ambulance or a bus. The APDD 10 may be mounted via bayonet slots 96 with a quick release rod 98 for ease of removal or installation. An IEC power inlet 100 and resettable on/off breaker switch 102 may be the same or similar to those parts used in FIG. 9 embodiments.

An embodiment, such as shown in FIG. 10 was tested by an independent laboratory and the State University of New York at Buffalo with 20 million active viral COVID inoculum load as input to the APDD 10. Testing at the output of the APDD 10 found no measurable viral particles at the outlet of the machine or at the inlet of the APDD 10.

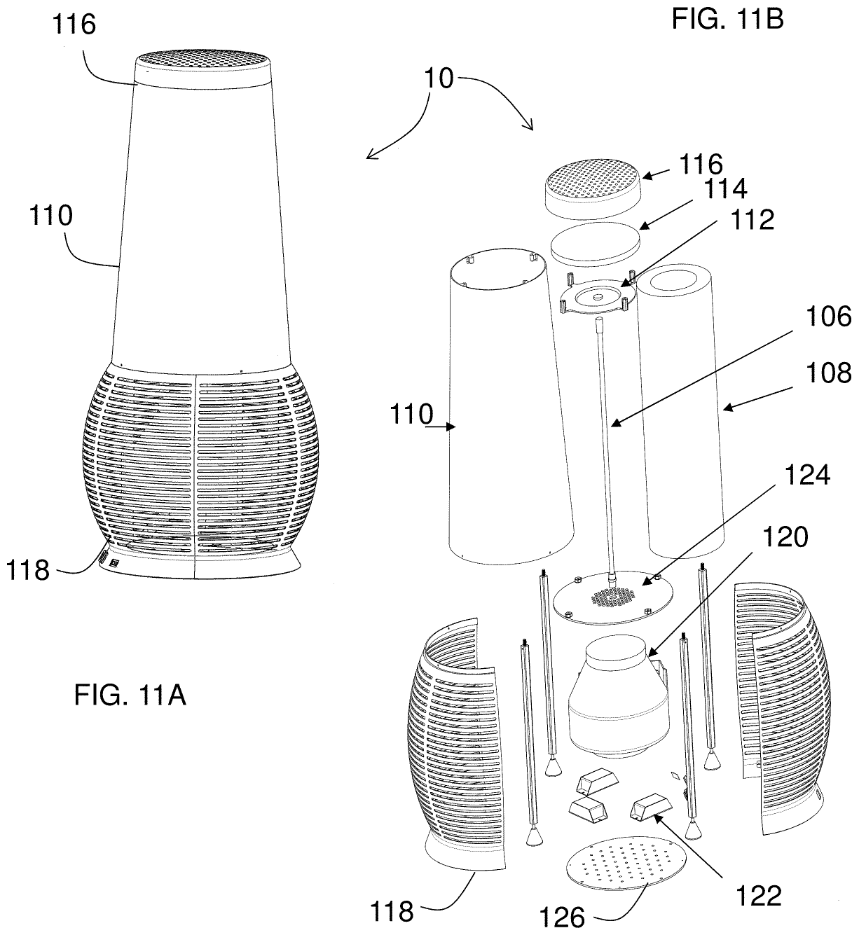

FIGS. 11A & 11B depict assembled and exploded views of other embodiments of the device 10, which may be used for residential room air purification or other purposes. The device 10 may a UV source 106 and a HEPA filter 108 mounted inside a cowl 110, which may be plastic, and may include a sealed reflector 112, HEPA top load cap 114, exhaust 116, air intake grill 118, miniature duct blower 120, motor controller and UV source 122. The blower 120 may be mounted to a HEPA/UV reflective base cap 124 by an isolator ring, such as silicon, which is not shown. Air may be drawn into the device 10 from the 360 degree intake grill 118 through the blower 120 and pushed through the base cap 124 and the HEPA filter 108 and exhausted out the top of the device 10 at 116. A safety cap 126 may be employed to prevent tampering with internal mechanisms.

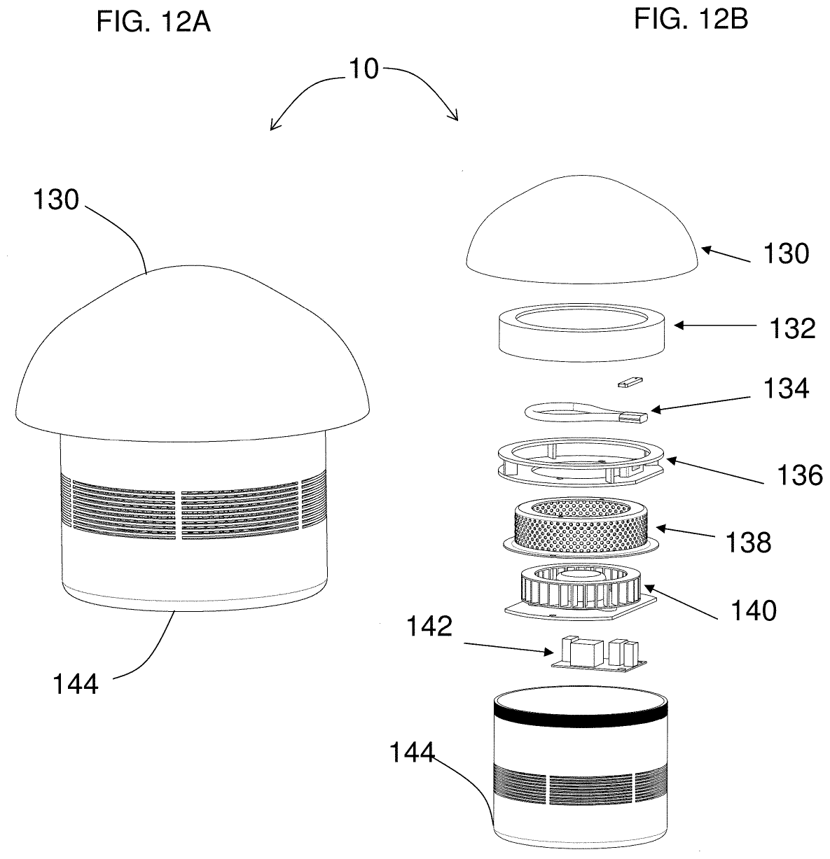

FIGS. 12A & 12B depict assembled and exploded views of other embodiments of the device 10 that may be employed as a desk top air purification device or other purposes. Air may be drawn in through a 360 degree intake shroud 130 and passed to a HEPA filter 138, which may be sealed by the UV source 134 and mount/HEPA top cap 136 underneath the UV reflector 132 through the cavity created by the outside case/exhaust vent 144 and HEPA filter 138 through the blower 140 which may be exhausted out through side vents 144. A motor controller 142 may be mounted in the device 10 proximate the blower 140 and configured to be controlled from a user interface on the device 10 and/or remotely.

As used herein, the term component is intended to be broadly construed as hardware, firmware, and/or a combination of hardware and software. It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware can be designed to implement the systems and/or methods based on the description herein.

Certain user interfaces have been described herein and/or shown in the figures. A user interface may include a graphical user interface, a non-graphical user interface, a text-based user interface, etc. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

The foregoing disclosure provides examples, illustrations and descriptions of the present invention, but is not intended to be exhaustive or to limit the implementations to the precise form disclosed. Modifications and variations are possible in light of the above disclosure or may be acquired from practice of the implementations. These and other variations and modifications of the present invention are possible and contemplated, and it is intended that the foregoing specification and the following claims cover such modifications and variations.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items and may be used interchangeably with “one or more.” Furthermore, as used herein, the term “set” is intended to include one or more items and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. An air purification device comprising:
an air inlet;
an air outlet;
an upstream section in air flow communication with the air inlet;
a downstream section in air flow communication with the air outlet;
a filter removably positioned between the upstream and downstream sections, the filter having an upstream surface and a downstream surface;
at least one ultraviolet (UV) illuminating source positioned in at least the upstream section proximate the filter to illuminate the upstream surface of the filter and kill viable pathogens that accumulate on the filter;
at least one ozone generating source positioned in the device to generate ozone in sufficient quantities to kill viable pathogens proximate the device;
wherein, the at least one UV illuminating source and the at least one ozone generating source are positioned such that the filter is removable without handling the sources.

2. The device of claim 1, where the device is part of a building air circulation system.

3. The device of claim 2, where the air circulation system is part of at least one of a heating, ventilation, and air conditioning system.

4. The device of claim 1, where the at least one illuminating source emits UV radiation at an energy level of at least 30 mW from 1 meter.

5. The device of claim 1, where the at least one illuminating source emits UV radiation at an energy level of at least 10,000 mW from 1 inch.

6. The device of claim 1, where the at least one UV illuminating source emits UV radiation in the 254 nm wavelength range.

7. The device of claim 1, where the ozone generating source is a UV source emitting UV radiation in the 182 nm wavelength range.

8. The device of claim 1, where the at least one ozone generating source is configured to produce ozone in sufficient quantities to provide for an ozone concentration of at least 3 ppm proximate the device.

9. The device of claim 1, where the at least one UV illuminating source includes a plurality of UV illuminating sources positioned to produce different illumination patterns on the filter.

10. The device of claim 1, where the UV illuminating and ozone generating sources are positioned to provide different air flow patterns in the upstream and downstream sections.

11. The device of claim 1, where the filter is composed of fiberglass.

12. The device of claim 1, where the upstream and downstream sections and filter are separable.

13. The device of claim 1, further comprising a fan position proximate to the downstream section to draw air through the upstream section and the filter.

14. The device of claim 1, where the at least one UV illuminating source and the at least one ozone generating source are further configured and positioned to reduce the pressure drop of air flowing from the air inlet to outlet through the filter.

15. The device of claim 1, where the at least one UV illuminating source is positioned to reduce viable pathogens proximate the filter to a non-hazardous concentration.

16. The device of claim 1, where the filter is at least one of a removeable cartridge and a removeable section of the device.

17. The device of claim 1, where the at least one UV illuminating source includes a plurality of UV illuminating sources positioned in the upstream section and a plurality of UV illuminating sources positioned in the downstream section.

18. A method of purifying air comprising:
providing an air purification device having
an air inlet,
an air outlet,
an upstream section in air flow communication with the air inlet,
a downstream section in air flow communication with the air outlet, and
a fan configured to draw air through the air inlet, upstream section, downstream section, and out of the air outlet;
positioning a filter removably between the upstream and downstream sections,
the filter having an upstream surface and a downstream surface;
drawing air, via the fan, through the air inlet, upstream section, filter, downstream section, and out of the air outlet;
illuminating, via at least one ultraviolet (UV) illuminating source positioned in at least the upstream section to kill viable pathogens that accumulate on the filter; and
generating, using at least one ozone generating source positioned in the device, ozone in sufficient quantities to kill viable pathogens proximate the device, where
the filter is removable without handling the at least one UV illuminating source and the at least one ozone generating source.

19. The method of claim 18, where
the at least one UV illuminating source is positioned to kill viable pathogens that accumulate on the upstream section proximate the filter.

20. An air purification device comprising:
an air inlet;
an air outlet;
an upstream section in air flow communication with the air inlet;
a downstream section in air flow communication with the air outlet;
a filter removably positioned between the upstream and downstream sections, the filter having an upstream surface, an interior, and a downstream surface;
a fan configured to draw air through the air inlet, upstream section, filter, downstream section, and out of the air outlet;
at least one ultraviolet (UV) illuminating source positioned in the upstream section proximate the filter to illuminate at least the upstream surface of the filter and kill viable pathogens that accumulate the upstream surface of the filter;
wherein, the at least one UV illuminating source are positioned such that the filter is removable without handling the at least one UV illuminating source.
